(19) United States
(12) Patent Application Publication
(10) Pub. No.: US 2020/0179300 A1
(43) Pub. Date: Jun. 11, 2020

(54) Topical Formulation Cures and Heals a Variety of Skin Conditions Including Ulcers, Decubitus Ulcers, Cancer, Abrasions and other Conditions and also accelerates the curing and healing of those Conditions

(71) Applicants: Joseph Urban, Richboro, PA (US); Shruti Thakorbhai Patel, Brookeville, MD (US); Charles Amsley Carmack, Key Colony Beach, FL (US)

(72) Inventors: Joseph Urban, Richboro, PA (US); Shruti Thakorbhai Patel, Brookeville, MD (US); Charles Amsley Carmack, Key Colony Beach, FL (US)

(21) Appl. No.: 16/350,586

(22) Filed: Dec. 10, 2018

(51) Int. Cl. A61K 31/045; A61K 9/00; A61P 17/02; A61K 9/06; A61K 47/44; A61K 33/30; A61K 47/46; A61K 31/7076; A61L 15/44; A61L 15/40; A61L 15/20; A61L 15/18; A61P 35/00

(52) U.S. Cl. CPC A61K 31/045; A61L 2300/416; A61P 17/02; A61K 9/06; A61K 47/44; A61K 33/30; A61K 47/46; A61K 31/7076; A61L 15/44; A61L 15/40; A61L 15/20; A61L 15/18; A61P 35/00; A61L 2300/106; A61L 2300/404; A61K 9/0014

(57) ABSTRACT

A formulation consisting of Thymol Iodide, in a Lanolin, Petrolatum, Zinc Oxide, Olive Oil base is applied to a topical skin lesion. Observed also and after discussions with patients, caregivers and patient relatives, it was discovered that a certain formulation consisting of an active pharmaceutical ingredient together with a specific base formulation promoted curing and accelerated healing of a variety of conditions. This formulation was examined for its superior effectiveness leading to the discovery found within this patent application.

Such a formulation will have a drastic reduction of healthcare costs because the topical application of this formulation will effect improvement, cure and accelerated healing resulting in no need for costly operations, hospitalizations, or long-term treatment.

### REFERENCES CITED

U.S. Patent Documents

| | | |
|---|---|---|
| 3,887,702 | June 1975 | Baldwin |
| 3,920,835 | November 1975 | Van Scott et al. |
| 3,984,566 | October 1976 | Van Scott et al. |
| 3,988,470 | October 1976 | Van Scott et al. |
| 4,105,783 | August 1978 | Yu et al. |
| 4,158,057 | June 1979 | Stanko |
| 4,197,316 | April 1980 | Yu et al. |
| 4,234,599 | November 1980 | Van Scott et al. |
| 4,246,261 | January 1981 | Van Scott et al. |
| 4,284,630 | August 1981 | Yu et al. |
| 4,294,852 | October 1981 | Wildnauer et al. |
| 4,351,835 | September 1982 | Stanko |
| 4,363,815 | December 1982 | Yu et al. |
| 4,415,576 | November 1983 | Stanko |
| 4,416,982 | November 1983 | Tsuda et al. |
| 4,451,482 | May 1984 | Cagen |
| 4,454,159 | June 1984 | Musher |
| 4,496,536 | January 1985 | Moller |
| 4,548,937 | October 1985 | Stanko |
| 4,615,697 | October 1986 | Robinson |
| 4,645,764 | February 1987 | Stanko |
| 4,663,166 | May 1987 | Veech |
| 4,696,917 | September 1987 | Lindstrom et al. |
| 4,725,586 | February 1988 | Lindstrom et al. |
| 4,734,276 | March 1988 | Ziegler |
| 4,812,479 | March 1989 | Stanko |
| 4,847,069 | July 1989 | Bissett et al. |
| 4,847,071 | July 1989 | Bissett et al. |
| 4,847,072 | July 1989 | Bissett et al. |
| 4,874,603 | October 1989 | Fratzer |

### Current U.S. Class: 514/532; 514/546

### Current International Class: A61K 31/70 (20060101); A61K 31/23 (20060101); A61K 31/215 (20060101); A61K 31/185 (20060101); A61K 31/19 (20060101); A61K 31/24 (20060101); A61K 31/22 (20060101); A61K 31/21 (20060101);

### Field of Search: 514/532; 514/546

### FIELD OF THE INVENTION

This invention relates to the field of medicine and wound care and more specifically to a Pharmaceutical Composition that when applied to a topical ulcer, and more specifically to a decubitus ulcer, a cancerous skin lesion, a topical breast abrasion, and other topical irritations, will provide accelerated curing and healing of such conditions.

### BACKGROUND OF THE INVENTION

The human body is covered with skin, the skin is a part of the integumentary system. The skin: waterproofs the body and prevents fluid loss; if unbroken blocks the penetration of pathogens into the body; is a receptor for the sense of touch; synthesizes vitamin D for the body; and protects the body from ultraviolet radiation.

There are a variety of lesions that occur on and in the skin. Some of those lesions which this application addresses are:
Ulcer—an open lesion of the skin resulting in tissue loss around the edges.
Decubitus Ulcer—a bedsore or pressure sore that is an open ulcerated wound caused by prolonged pressure on an area of the skin. If not treated properly, these ulcers can become infected resulting in tissue death.
Skin Cancer—a malignant growth on the skin usually one of the following three types: basal cell carcinoma; squamous cell carcinoma; and melanoma.
Skin Abrasion—a scraped surface or rubbed surface of the skin's superficial layers. Thus, a need exists for curing and healing skin problems and conditions.

### SUMMARY OF THE INVENTION

We are two practicing pharmacists and a pharmaceutical manufacturer of ointments and creams. In our practices we all have known and treated many patients with topical skin conditions and observed the healing rates for such conditions. We know first-hand that certain skin conditions never heal or heal incompletely over long periods of time. We have personally observed skin cancer lesions on the face, scalp, arms, legs which we believed to be squamous cell carcinoma or basal cell carcinoma. In these patients it appeared that the lesion would not heal, would bleed and appear to continue growing.

Observed also and after discussions with patients, caregivers and patient's relatives, it was discovered that a certain formulation consisting of an active pharmaceutical ingredient together with a specific base formulation promoted curing or accelerated healing of a variety of conditions. This formulation was examined for its superior effectiveness leading to the discovery found within this patent application.

Knowing that much of the effectiveness of this invention relies on its ability to demonstrate antimicrobial effectiveness, samples of the formulation were provided to an independent laboratory and ANTIMICROBIAL EFFECTIVENESS TESTING USP 40, NF 35<51>. The SUMMARY of the Study (#39412, Log #606718) states: “The preservative system utilized in the product tested should be evaluated according to the Criteria for Antimicrobial Effectiveness for Category 1-4 Products, USP 40, NP35, <51>, Antimicrobial Effectiveness Testing. Based on the Antimicrobial Effectiveness Test results, the product would PASS meeting the requirements for a Category 1 Preserved System. Based on the CFTA guidelines this product is considered well preserved.”

Such a formulation will have a drastic reduction on healthcare costs because the topical application of this formulation will effect cure or accelerated healing resulting in no need for costly operations, hospitalizations, or long-term treatment.

### DESCRIPTION OF CASES

The features of the invention believed to be novel and the elements characteristic of the invention are set forth with particularity in the following cases.

### Case 1—Decubitus Ulcer Treatment

A 76 year old male with senile dementia suffered from a decubitus ulcer about 2.5 cm in diameter not reaching the bone but with broken skin. The patient was bedridden and rotated periodically by caregivers but the bedsore still took hold and grew on his right buttock. He was treated with a mixture of Thymol Iodide in a Petrolatum, Zinc Oxide, Olive Oil and Lanolin Base.

Healing started at the peripheral portion of the ulcer within 48 hours with complete resolution of the ulcer within 7 days.

### Case 2—Skin Abrasion—Breast

A >45 year old female developed a 10 cm×10 cm skin abrasion under her left breast due to the breast rubbing against her chest wall under the breast. The skin was red, chafed, open, oozing and bleeding. The patient had tried nystatln ointment, triple antibiotic ointment, and hydrocortisone 1% cream all for 5 day periods of time each without any success. She was supplied with a mixture of Thymol Iodide in a Petrolatum, Zinc Oxide, Olive Oil and Lanolin Base and told to apply the mixture twice a day.

Healing started over the entire 10 cm×10 cm within 24 hours. The abrasion was completely healed within 5 days.

### Case 3—Skin Cancer—Face

A 70 year old male presented with a 3 mm Basal Cell Carcinoma lesion on his forehead. The lesion had been previously identified and treated by his dermatologist. He indicated that the lesion reappeared shortly after cryosurgery by the dermatologist and continued to peal repeatedly and bleed. The patient applied a mixture of Thymol Iodide in a Petrolatum, Zinc Oxide, Olive Oil and Lanolin Base to the lesion twice daily. Complete resolution occurred within 96 hours.

### Case 4—Skin Cancer—Leg

A 70 year old male presented with a 3.5 cm diameter Basal Cell Carcinoma lesion on his right leg. The lesion had been previously identified and treated by his dermatologist. He indicated that the lesion reappeared shortly after cryosurgery by the dermatologist and continued to peal repeatedly. The patient applied a mixture of Thymol Iodide in a Petrolatum, Zinc Oxide, Olive Oil and Lanolin Base to the lesion twice daily. Complete resolution occurred within 7 days.

### Case 5—Decubitus Ulcer Treatment

A 70 year old female with senile dementia suffered from a decubitus ulcer about 2 cm in diameter not reaching the bone but with broken skin. The patient was bedridden. She was treated with a mixture of Thymol Iodide in a Petrolatum, Zinc Oxide, Olive Oil and Lanolin Base.

Healing started at the peripheral portion of the ulcer within 48 hours with complete resolution of the ulcer within 7 days.

### Case 6—Accelerated Skin Growth

A 2 year old male bit into a live electrical line and the electric burned away a 2.5 cm diameter section of his lip on the left side of his face. Treatment was with a mixture of three antibiotics in a petrolatum base ointment with additives consisting of: a fatty acid fuel source for acetyl coenzyme A generation and an antioxidant. It is believed that antibiotics kept the skin from getting infected and the petrolatum with additives accelerated and increased the skin growth. All said, the skin began to grow too quickly and it was necessary to construct a prosthesis which hooked into the lip area of the re-growing burned skin site, and was held in place by a second part of the prosthesis wrapped around the ear. This device was needed for about 6 months to hold the accelerated skin growth from forming too small a lip.

### Case 7—Skin Cancer—Scalp

A 70 year old female presented with a 1.3 cm Basal Cell Carcinoma lesion on her scalp. The lesion had been previously identified and not yet treated. At this time the lesion was beginning to bleed in its central part. The patient applied a mixture of Thymol Iodide in a Petrolatum, Zinc Oxide, Olive Oil and Lanolin Base to the lesion twice daily. Healing started within 48 hours and noticeable resolution was observed at 7 days. Complete resolution took about 21 days.

### DETAILED DESCRIPTION OF THE INVENTION

This invention will now be described in detail.

The pharmaceutical topical preparation may be in an ointment, cream, gel, liquid, emulsion, semi-solid, or other form that permits contact with the surface of the skin. It may also be used on, within, or on top of a bandage, device or dressing. This invention consists of the following ingredients for the purposes so presented:
1—At least one antimicrobial/anti-infective agent that is safe and effective for the treatment of ulcerations, skin infections, wounds, cancer and other skin conditions. Such an agent was discovered to be Thymol Iodide C₂₀H₂₄I₂O₂ with a Molecular Weight of 550.23 in concentrations from 0.001% to 100.0% with the ideal concentration from 0.70% to 1.0%. Thymol Iodide has for its main purpose to provide an antiseptic, antibacterial, antifungal and other antimicrobial/antiviral environment. This environment is paramount to the regrowth of tissue and protection of the wound from a contaminated environment. Thymol Iodide also promotes the healing of ulcerations, wounds, and skin infection. Thymol iodide has also been used internally for healing cancerous pleural effusions but the inventiveness here is that it heals cancerous lesions externally on the skin as well when left in contact with the lesion.
2—A base substance that is used to contain the Thymol Iodide and provide protection for the skin. Such a base substance was determined to be Lanolin. In addition to being an ointment base, lanolin provides over 30 fatty acids which are converted into Acetyl Coenzyme A and effect an enhanced healing of the of ulcerations, skin infections, wounds, skin cancer and other skin conditions. The lanolin is also able to retain and absorb water from the environment and within the skin. The amount of Lanolin that can be used is from 0% to 100% with the ideal concentration from 30.8% to 60.8%. If lanolin is not used as a portion of the base, a replacement base preferably contains a fatty acid.
3—A base substance that Is used to contain the Thymol Iodide and provide protection for the skin. Such a base substance was determined to be Petrolatum. In addition to being an ointment base, Petrolatum provides an occlusive dressing over the skin. The amount of Petrolatum that can be used is from 0% to 100% with the ideal concentration from 24.0% to 54%. If petrolatum is not used as a portion of the base, a replacement base preferably provides an occlusive dressing to the skin.
4—A protective, astringent, antiseptic, substance that is preferably lethal to cancerous cells and offers protection for the skin. It was founded that Zinc Oxide is such a substance. The amount of Zinc Oxide that can be used is from 0% to 100% with the ideal concentration from 4% to 14%. If zinc oxide is not used the replacement substance should provide protective, astringent, antiseptic, and be lethal to cancer cells.
5—An emollient base substance that is used to contain the Thymol Iodide. Such an emollient base substance was determined to be Olive Oil. In addition to being an emollient base, Olive Oil provides fatty acids which are converted into Acetyl Coenzyme A and effect an enhanced healing of ulcerations, skin infections, wounds, skin cancer and other skin conditions. The amount of Olive Oil that can be used is from 0% to 100% with the ideal concentration from 0% to 10.7%. If Olive Oil is not used as a portion of the base, a replacement emollient base substance preferably contains fatty acids.
6—The complete finished product may be prepared by any suitable method or with any suitable equipment as know by those familiar with the art of Pharmacy and Manufacturing Pharmacy. The completed finished product may be prepared with use of heat or at room temperature without any heat.
7—To produce a batch of the finished product using heat, the following procedure was used: Heat a supply of Lanolin to 140° F. Heat a separate supply of Petrolatum to 137° F. Charge a 5 Liter stainless-steel mixing bowl with 170 G of Thymol Iodide. Add 1,070 G Olive Oil to the Thymol Iodide and mix high speed 3 minutes or until uniformly dispersed. Charge 7,800 G of heated Petrolatum into stainless-steel mixer bowl of Thoroughbred Mixer. Charge 1,800 G of Zinc Oxide into the Thoroughbred Mixer. Mix for 5 minutes or until uniformly dispersed. Add 9,160 G of heated Lanolin to the Thoroughbred Mixer. Mix for 5 minutes or until uniform. Add the Thymol Iodide-Olive Oil mix in the 5 Liter mixer bowl to the blend in the Thoroughbred Mixer. Mix for 5 minutes or until uniform. Allow to cool, transfer batch to filling machine and fill into final container.
8—To produce a batch of the finished product without using heat, the following procedure was used: Charge a 5 Liter stainless-steel mixing bowl with 170 G of Thymol Iodide. Add 1,070 G Olive Oil to the Thymol Iodide and mix high speed 3 minutes or until uniformly dispersed. Charge 7,800 G of Petrolatum into stainless-steel mixer bowl of Thoroughbred Mixer. Charge 1,800 G of Zinc Oxide into the Thoroughbred Mixer. Mix for 5 minutes or until uniformly dispersed. Add 9,160 G of Lanolin to the Thoroughbred Mixer. Mix for 5 minutes or until uniform. Add the Thymol Iodide-Olive Oil mix in the 5 Liter mixer bowl to the blend in the Thoroughbred Mixer. Mix for 5 minutes or until uniform. Transfer batch to filling machine and fill into final container.

We claim:

1. A topical formulation for the treatment of ulcerations, decubitus ulcers, skin infections, wounds, skin cancer and other skin conditions the pharmaceutical topical preparation may be in an ointment, cream, gel, liquid, emulsion, semi-solid, or other form that permits contact with the surface of the skin and it may also be used on, within, or on top of a bandage, device or dressing, said formulation containing Thymol Iodide C20H24I2O2 with a Molecular Weight of 550.23 in concentrations from 0.001% to 100.0% with the ideal concentration from 0.70% to 1.0%.

2. A formulation as described in claim 1 further comprising: A base substance that is used to contain the Thymol Iodide and provide protection for the skin, such a base substance is Lanolin providing over 30 fatty acids to the formulation said amount of Lanolin that can be used is from 0% to 100% with the ideal concentration from 30.8% to 60.8%.

3. A formulation as described in claim 1 further comprising: A base substance that is used to contain the Thymol Iodide and provide protection for the skin, such a base substance was determined to be Petrolatum which is an ointment base and provides an occlusive dressing over the skin, said amount of Petrolatum that can be used is from 0% to 100% with the ideal concentration from 24.0% to 54%.

4. A formulation as described in claim 1 further comprising: A protective, astringent, antiseptic, substance that offers some lethality to cancerous cells and offers protection for the skin, such a substance was founded to be Zinc Oxide, said amount of Zinc Oxide that can be used is from 0% to 100% with the ideal concentration from 4% to 14%.

5. A formulation as described in claim 1 further comprising: An emollient base substance that is used to contain the Thymol Iodide, such an emollient base substance was determined to be Olive Oil, said Olive Oil in addition to being an emollient base, provides fatty acids to the formulation which are converted into Acetyl Coenzyme A and effect an enhanced healing of ulcerations, skin infections, wounds, skin cancer and other skin conditions when applied to the skin with said amount of Olive Oil that can be used from 0% to 100% with the ideal concentration from 0% to 10.7%.

6. A formulation consisting of Thymol Iodide, Lanolin, Petrolatum, Zinc Oxide and Olive Oil prepared as a pharmaceutical topical preparation in an ointment, cream, gel, liquid, emulsion, semi-solid, or other form that permits contact with the surface of the skin and which may also be used on, within, or on top of a bandage, device or dressing.

7. A formulation as described in claim 6 further comprising: Thymol Iodide 0.85%, Lanolin 45.8%, Petrolatum 39.0%, Zinc Oxide 9.0%, Olive Oil 5.35% prepared as a pharmaceutical topical preparation in an ointment, cream, gel, liquid, emulsion, semi-solid, or other form that permits contact with the surface of the skin and which may also be used on, within, or on top of a bandage, device or dressing.

8. A formulation consisting of at least one antimicrobial/anti-infective agent that is safe and effective for the treatment of ulcerations, skin infections, wounds, cancer and other skin conditions and a pharmaceutical base that provides fatty acids and a protective, astringent, antiseptic, substance that is preferably lethal to cancerous cells and offers protection for the skin.

9. A formulation as described in claim 8 prepared as a pharmaceutical topical preparation in an ointment, cream, gel, liquid, emulsion, semi-solid, or other form that permits contact with the surface of the skin and which may also be used on, within, or on top of a bandage, device or dressing.
